SEQUENCE LISTING (7 sequences)

SEQ ID NO: 1 - DNA, 29 bp, Artificial Sequence
  misc_feature (1)..(29): primer
ttttttctagaactaggcatgggtctagc

SEQ ID NO: 2 - DNA, 27 bp, Artificial Sequence
  misc_feature (1)..(27): primer
tttttggatccccatcaatacttgttc

SEQ ID NO: 3 - DNA, 31 bp, Artificial Sequence
  misc_feature (1)..(31): primer
attacgagctcaagcttgatccatgcccttc

SEQ ID NO: 4 - DNA, 28 bp, Artificial Sequence
  misc_feature (1)..(28): primer
aatcggaattcaaatcttaggtgctttc

SEQ ID NO: 5 - DNA, 29 bp, Artificial Sequence
  misc_feature (1)..(29): primer
aatcgctcgagactaggcatgggtctagc

SEQ ID NO: 6 - DNA, 44 bp, Artificial Sequence
  misc_feature (1)..(44): primer
aaattggtaccgagctcaagcttgattttggttttaggaattag

SEQ ID NO: 7 - DNA, 35 bp, Artificial Sequence
  misc_feature (1)..(35): primer
aaattggtaccgagctcgacggtatcgataagctt

(19) United States
(12) Patent Application Publication
(10) Pub. No.: US 2002/0058340 A1
(43) Pub. Date: May 16, 2002

(54) Down-regulation of single genes and simultaneous down-regulation of multiple genes by nuclear localization of RNA transcripts

(76) Inventors: Thomas E. Clemente, Lincoln, NE (US); Paul E. Staswick, Lincoln, NE (US); Tony L. Buhr, Lincoln, NE (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 09/921,068

(22) Filed: Aug. 2, 2001

Related U.S. Application Data
(60) Provisional application No. 60/222,510, filed on Aug. 2, 2000.

(51) Int. Cl. C12N 15/87; A61K 48/00

(52) U.S. Cl. 435/455; 514/44

(57) ABSTRACT

A method is provided for gene suppression in eukaryotes by transformation with a recombinant construct containing a promoter, at least one antisense and/or sense nucleotide sequence for the gene(s) to be suppressed, wherein the nucleus-to-cytoplasm transport of the transcription products of the construct is inhibited. In one embodiment, nucleus-to-cytoplasm transport is inhibited by the absence of a normal 3′ UTR. The construct can optionally include at least one self-cleaving ribozyme. The construct can also optionally include sense and/or antisense sequences to multiple genes that are to be simultaneously down-regulated using a single promoter. Also disclosed are vectors, plants, animals, seeds, gametes, and embryos containing the recombinant constructs.

### CROSS-REFERENCE TO RELATED APPLICATIONS

This application claims the benefit of U.S. Provisional patent application Ser. No. 60/222,510, filed Aug. 2, 2000, which is herein incorporated by reference in its entirety for all purposes.

### BACKGROUND

The present invention is directed to a method for suppressing the expression of a gene or genes using nuclear localized RNA transcripts. Also, this invention is directed to the use of a single promoter to down-regulate more than one gene simultaneously.

There are currently at least four known methods for gene down-regulation or suppression in plants. One strategy is targeted gene disruption(s) or gene knockout(s) that relies on homologous recombination. “Knockout” refers to a DNA sequence that has been altered via any known means, for example, deletion, insertion, point mutation or rearrangement, so as to eliminate the function of the gene product. One strategy that generates a gene knockout in plants employs chimeric RNA/DNA oligonucleotides to generate site-specific mutations including gene knockouts (Beetham et al., (1999) Proc. Natl. Acad. Sci. USA, 96:8774). This technology requires specialized oligonucleotides and biolistic delivery that produces large numbers of undesirable off-type transgenics. It is also not clear whether the oligonucleotide-directed mutations are restricted to target genes.

Another strategy for gene down-regulation is expression of antisense transcripts, where the gene of interest has an antisense orientation between a promoter and an appropriate 3′ terminal sequence for nuclear export. As is well known, a cell manufactures protein by transcribing the DNA of the gene for that protein to produce RNA, which is then processed (e.g. by the removal of introns) into messenger RNA (mRNA) and finally translated by ribosomes into protein. This process may be inhibited by the presence in the cell of “antisense RNA”. Therefore, as used herein, the term “antisense RNA” means an RNA sequence which is complementary to a sequence of bases in a RNA: complementary in the sense that each base (or a majority of bases) in the antisense sequence (read in the 3′ to 5′ sense) is capable of pairing with the corresponding base (G with C, A with U) in the mRNA sequence read in the 5′ to 3′ sense. It is believed that this inhibition takes place by formation of a complex between the two complementary strands of RNA, preventing the production of protein. How this works is uncertain: the complex may interfere with further transcription, processing, transport or translation, or lead to degradation of the mRNA, or have more than one of these effects. Such antisense RNA may be produced in the cell by transformation with an appropriate DNA construct arranged to transcribe part of the antisense or anticoding strand (as opposed to the template strand) of the relevant gene (or of a DNA sequence showing substantial homology therewith).

The use of this technology to down-regulate the expression of specific plant genes has been described, for example in European Patent Publication No 271988. Reduction of gene expression by the use of antisense RNA has led to a change in the phenotype of the plant: either at the level of gross visible phenotypic difference e.g. lack of lycopene synthesis in the fruit of tomato leading to the production of yellow rather than red fruit, or at a more subtle biochemical level e.g. change in the amount of polygalacturonase and reduction in depolymerisation of pectins during tomato fruit ripening (U.S. Pat. Nos. 5.073,676, 5,107,065, 5,569,831; Smith et al, (1988) Nature, 334, 724-726; Smith et al, (1990) Plant Mol. Biol. 14, 369-380). Thus, antisense RNA is one method used in achieving down-regulation of gene expression in plants. However, antisense down-regulation is often leaky since there is frequently a reduction of the gene product but not always a complete shut-down of the gene product.

In some instances, antisense constructs have been combined with ribozymes. Ribozymes are catalytic RNA molecules that can promote specific biochemical reactions without the need for auxiliary proteins. U.S. Pat. No. 5,814,500 discloses a construct containing an antisense sequence flanked on both ends by a U1 snRNA stem loop structure. The stem loop structure is believed to give the antisense construct enhanced stability by being resistant to nuclease activity and by being enriched in the nucleus. In one embodiment, the construct contains a ribozyme sequence which cleaves a target RNA and is located between stem loop structures which were placed on each end of the construct. The patent teaches the use of this construct in animal cells. Likewise, U.S. Pat. No. 5,908,779 discloses mammalian cells containing a promoter, an antisense sequence and a cis-ribozyme. The use of antisense construct harboring cis-acting ribozymes has not been successfully applied to plants.

A third strategy for gene down-regulation is co-suppression. Co-suppression can occur when the gene of interest is expressed in the sense orientation between a promoter and an appropriate 3′ terminal sequence for nuclear export. Co-suppression results in reduced expression of the transgene as well as the endogenous gene. Insertion of a related gene or promoter into a plant can induce rapid turnover of homologous endogenous transcripts, a process believed to have many similarities to the mechanism responsible for antisense RNA inhibition. The effect depends on sequence identity between the transgene and the endogenous gene. Some cases of co-suppression resemble RNA interference (the experimental silencing of genes by the introduction of double-stranded RNA), as RNA seems to be both an important initiator and a target in these processes. Various regulatory sequences of DNA can be altered (promoters, polyadenylation signals, post-transcriptional processing sites) or used to alter the expression levels (enhancers and silencers) of a specific mRNA.

The fourth strategy for gene down-regulation or suppression is RNA interference. Introduction of double-stranded RNA induces potent and specific gene silencing, a phenomenon called RNA interference or RNAi. In RNAi, inverted repeats, which presumably form strong secondary structure in vivo, cause genetic down-regulation. (Sharp et al. (2000) Science 287:2431-2433). The mechanism of how RNAi results in the loss of the targeted homologous mRNA is still not well understood. The RNAi strategy is effective for gene down-regulation in plants, and is distinct from the method using expression of antisense transcripts because the base pairing in RNAi occurs intramolecularly.

Importantly, current models generally indicate that the mechanisms for the above down-regulation strategies originate in the cytoplasm of the cell. (Marx, J. (2000) Science 288:1370-1372; Wolfe et al., (1999) Science 286:481-486; Waterhouse et al. (1998) PNAS 95:13959-13964). Cytoplasmic localization of transcripts presumably causes dilution of the transcripts as opposed to nuclear localization. Thus, there remains a need in the art for methods useful in the suppression and down-regulation of genes which uses a nuclear localization strategy of RNA transcripts in order to increase the efficacy of gene down-regulation.

### SUMMARY

One aspect of the present invention, therefore, provides a method by which expression of genes can be suppressed (down regulated). Described herein are constructs and methods which allow the nuclear localization of RNA. The presence of high concentrations of either sense or antisense RNA within the nucleus results in the suppression of the gene or genes of interest.

Another aspect provides a method for suppressing gene expression in a eukaryotic cell comprising transforming the cell with a recombinant construct comprising, a promoter functional in the cell operatively linked to a sense nucleotide sequence of a gene to be suppressed, wherein nucleus-to-cytoplasm transport of transcription products of the construct is inhibited. In one embodiment, the construct can contain sequences for the suppression of multiple genes.

A further aspect provides a method for suppressing gene expression in a eukaryotic cell comprising transforming the cell with a recombinant construct comprising, a promoter functional in the cell operatively linked to a plurality of antisense nucleotide sequences of a gene or genes to be suppressed, wherein nucleus-to-cytoplasm transport of transcription products of the construct is inhibited.

Still another aspect provides a method for suppressing expression of a gene in a plant cell comprising, transforming the plant cell with a recombinant construct comprising, a promoter functional in said plant; and an antisense nucleotide sequence for the gene to be suppressed, wherein nucleus-to-cytoplasm transport of transcription products of the construct is inhibited.

Yet another aspect provides a recombinant vector comprising a promoter functional in a eukaryotic cell operatively linked to at least one sense nucleotide sequence of at least one gene to be suppressed, wherein nucleus-to-cytoplasm transport of transcription products of said at least one nucleotide sequence is inhibited.

An additional aspect provides a recombinant vector comprising a promoter functional in a eukaryotic cell operatively linked to a plurality of antisense nucleotide sequences of at least one gene to be suppressed, wherein nucleus-to-cytoplasm transport of transcription products of said at least one nucleotide sequence is inhibited.

Another aspect provides a recombinant vector comprising a promoter functional in a plant; and at least one antisense sequence for at least one gene to be suppressed; wherein nucleus-to-cytoplasm transport of transcription products of said at least one nucleotide sequence is inhibited.

Still another aspect provides, a eukaryotic cell whose genome includes a recombinant construct comprising a promoter functional in the eukaryotic cell operatively linked to at least one sense nucleotide sequence of at least one gene to be suppressed, wherein nucleus-to-cytoplasm transport of transcription products of the construct is inhibited.

An additional aspect provides, a eukaryotic cell whose genome includes a recombinant construct comprising a promoter functional in the eukaryotic cell operatively linked to a plurality of antisense nucleotide sequences of at least one gene to be suppressed, wherein nucleus-to-cytoplasm transport of transcription products of the construct is inhibited.

A further aspect provides, a plant cell whose genome includes a recombinant construct comprising, a promoter functional in said plant cell operatively linked to at least one antisense sequence for at least one gene to be suppressed; wherein nucleus-to-cytoplasm transport of transcription products of the construct is inhibited.

In still further aspects, any of the aforementioned constructs which contain more than one sequence can comprising a combination of sense and antisense sequences.

Additional aspects include, plants and animals comprising any of the aforementioned cells or constructs; and seed, gametes, embryos, progeny and uniform populations obtained from such plants or animals.

In any of the aforementioned aspects, the inhibition of nucleus-to-cytoplasm transport can be due to the absence of appropriate 3′ terminal sequences (3′ UTR), which are typically necessary for nucleus-to-cytoplasm transport of transcription products, or the presence of 3′ terminal sequences that prevent or avoid nuclear-to-cytoplasm transport of transcription products.

In additional aspects, any of the aforementioned constructs can include at least one self cleaving ribozyme.

In still further aspects, any of the aforementioned promoters can be constitutive, inducible, tissue specific, developmentally regulated, or a suitable combination such as an inducible, tissue specific promoter.

### BRIEF DESCRIPTION OF THE DRAWINGS

These and other features, aspects, and advantages of the present invention will become better understood with regard to the following description, appended claims and accompanying figures where:

FIG. 1 shows the arrangement of pertinent DNA sequences for the binary vectors pPTN102, pPTN110, pPTN111, pPTN112 and pPTN113. The sizes of the different regions are not to scale. Abbreviations used are as follows: LB=left border, hpt=hygromycin phosphotransferase ORF, Pnos=nopaline synthase promoter, E35S=cauliflower mosaic virus 35S enhancer, TEV=tobacco etch virus translational leader, T35S=cauliflower mosaic virus 35S 3′ terminal sequence, RB=right border, nptII=neomycin phosphotranferase ORF, P35S=cauliflower mosaic virus promoter, RZ=ribozyme, GUS sense=GUS ORF in sense orientation, anti GUS=GUS ORF in antisense orientation.

FIG. 2 shows the arrangement of pertinent DNA sequences for the binary vectors pPTN166, pPTN167, pPTN170 and pPTN326 that a single gene knockout constructs for soybean. The sizes of different regions are not to scale. Abbreviations are the same as for FIG. 1 with the following additions: bar=glufosinate resistance gene, FAD2-1=FAD2-1 gene in sense orientation, anti-FAD2-1=FAD2-1 gene in antisense orientation, PhP=Phaseolin promoter.

FIG. 3 shows the arrangement of pertinent DNA sequences for the binary vectors pPTN300 and pPTN303 that are dual gene knockout constructs for soybean. The sizes of different regions are not to scale. Abbreviations are the same as for FIGS. 1 and 2 with the following additions: B-C-=β-conglycinin promoter, FatB=FatB gene in sense orientation.

FIG. 4 shows a photograph of nuclei-enriched fraction from tobacco. The color picture on the left was stained with propidium iodide. The phase contrast picture on the right is of the same region.

FIG. 5 shows a Northern hybridization for transgenic tobacco harboring pPTN102 (total RNA, lane 1 and nuclei RNA, lane 2), pPTN110 (total RNA, lane 3 and nuclei RNA, lane 4), pPTN111 (total RNA, lane 5 and nuclei RNA, lane 6), pPTN112 (total RNA, lane 7 and nuclei RNA, lane 8), and pPTN113 (total RNA, lane 9 and nuclei RNA, lane 10). The upper portion was probed with the TEV-GUS sequence from pPTN102 and the lower portion was probed with rDNA.

FIG. 6 shows photographs of in situ hybridizations. The pictures in the column on the left were taken using the red channel to show nuclei. The pictures in the center column were taken using the green channel to show probe hybridizations. The pictures in the column on the right are the red and green channels combined. The rows of pictures are arranged to show tobacco sections harboring pPTN111 or pPTN112 probed with either the GUS sense probe or the GUS antisense probe.

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications and other references cited in this application are herein incorporated by reference in their entirety as if each individual publication, patent, patent application or other reference were specifically and individually indicated to be incorporated by reference.

The following abbreviations and definitions are used herein.

GUS=β-glucuronidase

ORF=open reading frame

TEV=tobacco etch virus

35S=CaMV35S=cauliflower mosaic virus 35S

FAD2-1=An omega-6-desaturase that catalyzes desaturation at the omega 6 carbon (sixth carbon from the end).

ddH₂O=deionized, distilled water

MOPS=2-(N-Morpholino)-2-hydroxypropanesulfonic acid

EDTA=ethylenediaminetetraacetic acid

SDS=sodium dodecyl sulfate

SSC=standard saline citrate (20× SSC=3 M NaCl, 0.3 M citrate.2H2O, pH=7)

TE=10 mM Tris, 1 mM EDTA

DIG=digoxigenin

1× TBS=20 mM Tris, 0.5M NaCl, pH=7.5

TTBS=0.05% Tween 20, 1× TBS

BSA=bovine serum albumin

FITC=fluorescein isothiocyanate

LB=Luria-Bertani medium=10 g bacto-tryptone, 5 g bacto-yeast extract, 10 g NaCl in one liter water, pH=7.

T-DNA=that part of the Agrobacterium Ti plasmid that is incorporated into the genome of infected plant cells.

3′ UTR=3′ untranslated region or sequence of a gene or the transcription product of that gene.

The term “expression” in the context of a gene or polynucleotide involves the transcription of the gene or polynucleotide into RNA. The term can also, but not necessarily, involve the subsequent translation of the RNA into polypeptide chains and their assembly into proteins.

As used herein, “expression vector” means a vector that promotes transcription of a gene or polynucleotide. When placed in a suitable host cell, transcripts from an expression vector can result in synthesis of a protein or polypeptide.

As used herein, “polynucleotide” and “oligonucleotide” are used interchangeably and mean a polymer of at least 2 nucleotides joined together by phosphodiester bonds and may consist of either ribonucleotides or deoxyribonucleotides.

As used herein, “sequence” means the linear order in which monomers occur in a polymer, for example, the order of amino acids in a polypeptide or the order of nucleotides in a polynucleotide.

As used herein, “peptide” and “protein” are used interchangeably and mean a compound that consists of two or more amino acids that are linked by means of peptide bonds.

As used herein, “organelle” means any discrete structure in a cell that is adapted and/or specialized for the performance of one or more vital functions and can include plastids and mitochondria.

As used herein, “recombinant construct” is defined either by its method of production or its structure. In reference to its method of production, e.g., a product made by a process, the process is use of recombinant nucleic acid techniques, e.g., involving human intervention in the nucleotide sequence, typically selection or production. Alternatively, it can be a sequence comprising fusion of two nucleic acid sequences which are not naturally contiguous to each other, but is meant to exclude products of nature, e.g., naturally occurring mutants.

The present invention is directed to the suppression of gene expression by use of a DNA construct comprising a promoter, and a sense or antisense sequence for the gene or genes to be suppressed wherein the normal nucleus-to-cytoplasm transport of the RNA transcript of the construct is inhibited. In one embodiment, transport is inhibited due to the absence of the normal 3′ terminal sequence. As used herein, a “normal” 3′ terminal sequence (3′ UTR) is a sequence found at the 3′ end of a gene that is involved in nuclear-to-cytoplasm transport of transcription products of the gene. The absence of a “normal” 3′ UTR from a gene or the transcription product of that gene, or the replacement of a “normal” 3′ UTR with an appropriate alternative sequence will prevent or inhibit nucleus to cytoplasm transport of the transcription product (Eckner et al., EMBO J., 10:3513-3522; Huang and Carmichael (1996) Molec. Cell. Biol., 16:1534-1542). In another embodiment, the construct also includes a self-cleaving ribozyme. It should be recognized that the suppression of gene expression need not be complete. Rather, the rate of translation of the protein encoded by the gene to be suppressed need only be decreased when compared to the rate of expression observed in the absence of the construct. In one embodiment, the rate of expression is decreased by at least 10%. In another embodiment, the rate of expression is decreased by at least 50% and in yet another embodiment, the rate of expression is decreased at least 75%. In still another embodiment, the rate of expression is decreased at least 95%.

The present invention is useful for altering physiological processes within a cell or tissue. For example, and without limitation, the present invention can be used to alter the patterns of enzymes produced which in turn can alter biochemical pathways resulting in the altered accumulation of the end products of such pathways. One non-limiting example is the type of fatty acids produced by the plant. Examples of other physiological processes which may be altered using the present invention include, flowering, and the ripening, spoilage of fruit and the fatty acid composition of meat and milk.

The present invention also has therapeutic uses such as the suppression of undesirable genes. These genes may be native genes or can be exogenous genes such as those due to viral or retroviral infection. Therefore, the present invention can be used to resist or treat such infections.

Any promoter that functions in the chosen host may be used in practicing the present invention. The promoter may be homologous or heterologous to the sequence controlled. Promoters useful in the present invention include those that confer appropriate cellular and temporal specificity of expression. Such promoters include those that are constitutive or inducible, environmentally-regulated, developmentally-regulated, cell-specific, or tissue-specific. In plants, often-used constitutive promoters include the CaMV 35S promoter (Odell et al. (1985) Nature 313: 810), the enhanced CaMV 35S promoter, the Figwort Mosaic Virus (FMV) promoter (Richins et al. (1987) NAR 20: 8451), the mannopine synthase (mas) promoter, the nopaline synthase (nos) promoter, and the octopine synthase (ocs) promoter.

Useful inducible promoters in plants include heat-shock promoters (Ou-Lee et al. (1986) Proc. Natl. Acad. Sci. USA 83: 6815; Ainley et al. (1990) Plant Mol. Biol. 14: 949), a nitrate-inducible promoter derived from the spinach nitrite reductase gene (Back et al. (1991) Plant Mol. Biol. 17:9), hormone-inducible promoters (Yamaguchi-Shinozaki et al. (1990) Plant Mol. Biol. 15: 905; Kares et al. (1990) Plant Mol. Biol. 15: 905), and light-inducible promoters associated with the small subunit of RuBP carboxylase and LHCP gene families (Kuhlemeier et al. (1989) Plant Cell 1: 471; Feinbaum et al. (1991) Mol. Gen. Genet. 226: 449; Weisshaar et al. (1991) EMBO J. 10: 1777; Lam and Chua (1990) Science 248: 471; Castresana et al. (1988) EMBO J. 7: 1929; Schulze-Lefert et al. (1989) EMBO J. 8: 651).

Examples of useful tissue-specific, developmentally-regulated promoters in plants include fruit-specific promoters such as the E4 promoter (Cordes et al. (1989) Plant Cell 1:1025), the E8 promoter (Deikman et al. (1988) EMBO J. 7: 3315), the kiwi fruit actinidin promoter (Lin et al. (1993) PNAS 90: 5939), the 2A11 promoter (Houck et al., U.S. Pat. No. 4,943,674), and the tomato pZ130 promoter (U.S. Pat. Nos. 5,175, 095 and 5,530,185); the β-conglycinin 7S promoter (Doyle et al. (1986) J. Biol. Chem. 261: 9228; Slighton and Beachy (1987) Planta 172: 356), and seed-specific promoters (Knutzon et al. (1992) Proc. Natl. Acad. Sci. USA 89: 2624; Bustos et al. (1991) EMBO J. 10: 1469; Lam and Chua (1991) J. Biol. Chem. 266: 17131; Stayton et al. (1991) Aust. J. Plant. Physiol. 18: 507). Fruit-specific gene regulation is discussed in U.S. Pat. No. 5,753,475. Other useful seed-specific promoters include, but are not limited to, the napin, phaseolin, zein, soybean trypsin inhibitor, 7S, ADR12, ACP, stearoyl-ACP desaturase, oleosin, Lasquerella hydroxylase, and barley aldose reductase promoters (Bartels (1995) Plant J. 7: 809-822), the EA9 promoter (U.S. Pat. No. 5,420,034), and the Bce4 promoter (U.S. Pat. No. 5,530,194). Useful embryo-specific promoters include the corn globulin 1 and oleosin promoters. Useful endosperm-specific promoters include the rice glutelin-1 promoter, the promoters for the low-pI α-amylase gene (Amy32b) (Rogers et al. (1984) J. Biol. Chem. 259: 12234), the high-pI α-amylase gene (Amy 64) (Khurseed et al. (1988) J. Biol. Chem. 263: 18953), and the promoter for a barley thiol protease gene (“Aleurain”) (Whittier et al. (1987) Nucleic Acids Res. 15: 2515).

Other promoters known to control the expression of genes in prokaryotic or eukaryotic cells can be used and are known to those skilled in the art. For example, the casein promoter can be used to direct expression of foreign protein(s) in the milk. Promoter hybrids can also be constructed to enhance transcriptional activity (Hoffman, U.S. Pat. No. 5,106,739), or to combine desired transcriptional activity and tissue specificity.

One embodiment of the present invention involves the use of antisense sequences. Antisense sequences can be produced by reversing the orientation of the transcribed region of a gene or polynucleotide sequence whose suppression is desired. When operatively coupled to a suitable transcriptional promoter such as those discussed above, a transcript of the antisense DNA strand is produced. The production and use of antisense DNA is well known in the art and can be found, for example, in Green et al., (1986) Annu. Rev. Biochem. 55:569. The transcript of the antisense DNA is antisense RNA. Without being bound by theory, it is believed that an individual antisense RNA molecule may hybridize with a complementary “sense” mRNA molecule to form an RNA-RNA duplex. Such a duplex may prevent the sense mRNA molecule from being translated, effectively suppressing production of the ultimate gene product, a protein. The presence of RNA-RNA duplexes may also initiate a sequence-specific RNA degradation pathway with the antisense molecules and/or the RNA-RNA duplexes playing a role in initiating the degradation pathway, and both sense and antisense molecules serving as specific targets for degradation. Formation of RNA-RNA duplexes is believed to be most efficient when the sense and antisense RNAs are retained in close proximity to each other. One advantage of the present invention is that the antisense RNA is not transported out of the nucleus into the cytoplasm. This maintains a relatively high concentration of antisense RNA within the nucleus and thereby may increase the likelihood that RNA-RNA duplexes will form before the sense mRNA is transported to the cytoplasm where translation can occur. It will be apparent to one of ordinary skill in the art, that the antisense transcript need not encompass the entire gene or polynucleotide sequence, but may be a fragment which hybridizes to only a portion of the sense RNA. The antisense transcript should be of sufficient length to allow specificity in binding to the target (sense) transcript. In general, the antisense transcript should be at least 10 bases long, although the presence of rare sequences may allow the use of shorter antisense transcripts.

As with antisense sequences, sense sequences used in the practice of the present invention need not encompass the entire gene. Rather, the sense sequences used can include only a portion of the gene. In one embodiment, the sense sequences include the coding portion of the gene. In another embodiment, the sequences are at least about 10 bases long.

An additional embodiment involves the use of a self-cleaving ribozyme, preferably located downstream to the sense or antisense sequence of the construct. Ribozymes are catalytic RNA molecules that can promote specific biochemical reactions without the need for auxiliary proteins. Reactions catalyzed by ribozymes can be either intramolecular or intermolecular. Examples of intramolecular reactions are self-splicing or self-cleaving reactions while intermolecular reactions involve other RNA molecules as substrates and more closely approximate true enzymatic reactions where the enzyme is unchanged after each reaction. In the present invention, a self-cleaving ribozyme is used. In one embodiment, the self-cleaving ribozyme is the RZ ribozyme described in U.S. Pat. No. 5,908,779 and Liu and Carmichael (1994) Molec. Biotechnol. 2:107. In one embodiment, the ribozyme replaces the 3′ terminal sequence normally found in RNA to be transported to the cytoplasm for translation. In addition, the ribozyme acts to cleave itself from the transcript produced creating a free transcript and further insuring that the transcript remains within the nucleus.

In addition, the present invention can also employ constructs that result in RNA with secondary structure (RNAi). An example of such secondary structure that induces RNAi would be encompassed in a construct that expresses a single transcript that has both sense and complementary antisense sequences from the target gene or genes.

optionally, the construct may also include an enhancer. Enhancers are DNA sequences found in eukaryotes that increase the rate of transcription of genes present on the same molecule, but which do not have promoter activity. An enhancer can be placed upstream or downstream of a promoter or in forward or reverse orientation without loss of activity.

The constructs of the present invention are produced using methods well known to those of ordinary skill in the art which can be found, for example, in standard texts such as Sambrook et al., Molecular Cloning, 2nd ed., Cold Spring Harbor Laboratory Press, 1989 and Ausubel, et al., Short Protocols in Molecular Biology, 3rd ed., Wiley & Sons, 1995. In general, recombinant constructs are produced by a series of restriction enzyme digestions and ligation reactions which result in the desired sequences being incorporated into a plasmid. If suitable restriction sites are not available, then synthetic oligonucleotide adapters or linkers can be used as is known to those skilled in the art and described in the references cited above. The DNA constructs are assembled such that the promoter, sense DNA and/or antisense DNA and ribozyme are operatively linked. A nucleic acid sequence is operably linked when it is placed into a functional relationship with another nucleic acid sequence. For example, a promoter is operably linked to a sequence if it affects the transcription of the sequence; or a ribosome binding site is operably linked to a coding sequence if it is positioned so as to facilitate translation.

It will be apparent to those of ordinary skill in the art, that the precise restriction enzymes, linkers and/or adaptors required as well as the precise reaction conditions will vary with the sequences and cloning vectors used. The assembly of DNA constructs, however, is routine in the art and can be readily accomplished by the skilled technician without undue experimentation. Non-limiting illustrations of the assembly of DNA constructs useful in the present invention can be found in the examples that follow.

The DNA construct can then be placed into a suitable vector to transform a host cell. The vector can be either a cloning vector or an expression vector. A cloning vector is a self-replicating DNA molecule that serves to transfer a DNA segment into a host cell. The three most common types of cloning vectors are bacterial plasmids, phages, and other viruses. An expression vector is a cloning vector designed so that a coding sequence inserted at a particular site will be transcribed into mRNA. Both cloning and expression vectors contain nucleotide sequences that allow the vectors to replicate in one or more suitable host cells. In cloning vectors, this sequence is generally one that enables the vector to replicate independently of the host cell chromosomes, and also includes either origins of replication or autonomously replicating sequences. Various bacterial and viral origins of replication are well known to those skilled in the art and include, but are not limited to, the pBR322 plasmid origin, the 2μ plasmid origin, and the SV40, polyoma, adenovirus, VSV and BPV viral origins. Ausubel et al., ed., Short Protocols in Molecular Biology, 3rd ed., Wiley & Sons, 1995.

Vectors can and usually do contain a selection gene or selection marker. Typically, this gene encodes a protein necessary for the survival or growth of the host cell transformed with the vector. Examples of suitable selection markers include dihydrofolate reductase (DHFR) or neomycin resistance for eukaryotic cells and tetracycline or ampicillin resistance for E. coli. Selection genes in plants include genes that confer resistance to bleomycin, gentamycin, glyphosate, hygromycin, kanamycin, methotrexate, phleomycin, phosphinotricin, spectinomycin, streptomycin, sulfonamide and sulfonylureas. Maliga et al., Methods in Plant Molecular Biology, Cold Spring Harbor Laboratory Press, 1995, p. 39.

In addition, vectors can also contain marker sequences. Suitable markers include, but are not limited to, alkaline phosphatase (AP), myc, hemagglutinin (HA), β-glucuronidase (GUS), luciferase, and green fluorescent protein (GFP).

A further embodiment of the present invention relates to transformed host cells containing constructs of the present invention. The host cell can be a higher eukaryotic cell, such as a plant or animal cell, or a lower eukaryotic cell such as a yeast cell. Introduction of the construct into the host cell can be accomplished by a variety of methods including calcium phosphate transfection, DEAE-dextran mediated transfection, Polybrene, protoplast fusion, liposomes, direct microinjection into the nuclei, scrape loading, and electroporation. In plants, a variety of different methods can be employed to introduce transformation/expression vectors into plant protoplasts, cells, callus tissue, leaf discs, meristems, etc., to generate transgenic plants. These methods include, for example, Agrobacterium-mediated transformation, particle gun delivery, microinjection, electroporation, polyethylene glycol-mediated protoplast transformation, liposome-mediated transformation, etc. (reviewed in Potrykus (1991) Annu. Rev. Plant Physiol. Plant Mol. Biol. 42: 205). In one embodiment, plant cells are transformed using Agrobacterium-mediated transformation and a binary vector system in which the vectors were introduced into the Agrobacterium by triparental matings (see, Maliga et al., Methods in Plant Molecular Biology, Cold Spring Harbor Laboratory Press, 1995, Sec. 4).

Transgenic animals can be produced by the injection of the polynucleotides of the present invention into the pronucleus of a fertilized oocyte, by transplantation of cells, preferably undifferentiated cells, into a developing embryo to produce a chimeric embryo, transplantation of a nucleus from a recombinant cell into an enucleated embryo or activated oocyst, or by any other method capable of producing a transgenic animal. Methods for the production of transgenic animals can be found in a number of references including, for example, U.S. Pat. No. 4,873,191; Rudolph (1999) Trends Biotechnol., 17:367-374; Dalrymple et al. (1998) Biotechnol. Genet. Eng. Rev., 15:33-49; Colman (1998) Biochem. Soc. Symp., 63:141-147; Wilmut et al., (1997) Nature 385:810-813; Wilmut et al. (1998) Reprod. Fertil. Dev., 10:639-643; Perry et al. (1993) Transgenic Res., 2:125-133; Hogan et al., Manipulating the Mouse Embryo, 2nd ed., Cold Spring Harbor Laboratory Press, 1994; and references cited therein. Of course, gametes, seeds, embryos, progeny and hybrids of plants or animals containing constructs of the present invention produced by traditional breeding methods are also included within the scope of the present invention.

In general, transgenic plants can be produced by transforming plant cells with a DNA construct as described above via any of the foregoing methods; selecting plant cells that have been transformed on a selective medium; regenerating plant cells that have been transformed to produce differentiated plants; and selecting a transformed plant that expresses the construct at a level sufficient to result in gene suppression.

Specific methods for transforming a wide variety of dicots and obtaining transgenic plants are well documented in the literature (Gasser and Fraley (1989) Science 244: 1293; Fisk and Dandekar (1993) Scientia Horticulturae 55: 5; Christou (1994) Agro Food Industry Hi Tech, p. 17; and the references cited therein).

Examples of successful transformation and plant regeneration in monocots are as follows: asparagus (Asparagus officinalis; Bytebier et al. (1987) Proc. Natl. Acad. Sci. USA 84: 5345); barley (Hordeum vulgarae; Wan and Lemaux (1994) Plant Physiol. 104: 37); maize (Zea mays; Rhodes et al. (1988) Science 240: 204; Gordon-Kamm et al. (1990) Plant Cell 2: 603; Fromm et al. (1990) Bio/Technology 8: 833; Koziel et al. (1993) Bio/Technology 11: 194); oats (Avena sativa; Somers et al. (1992) Bio/Technology 10: 1589); orchardgrass (Dactylis glomerata; Horn et al. (1988) Plant Cell Rep. 7: 469); rice (Oryza sativa, including indica and japonica varieties; Toriyama et al. (1988) Bio/Technology 6: 10; Zhang et al. (1988) Plant Cell Rep. 7: 379; Luo and Wu (1988) Plant Mol. Biol. Rep. 6: 165; Zhang and Wu (1988) Theor. Appl. Genet. 76: 835; Christou et al. (1991) Bio/Technology 9: 957); rye (Secale cereale; De la Pena et al. (1987) Nature 325: 274); sorghum (Sorghum bicolor; Cassas et al. (1993) Proc. Natl. Acad. Sci. USA 90: 11212); sugar cane (Saccharum spp.; Bower and Birch (1992) Plant J. 2: 409); tall fescue (Festuca arundinacea; Wang et al. (1992) Bio/Technology 10: 691); turfgrass (Agrostis palustris; Zhong et al. (1993) Plant Cell Rep. 13: 1); and wheat (Triticum aestivum; Vasil et al. (1992) Bio/Technology 10: 667; Weeks et al. (1993) Plant Physiol. 102: 1077; Becker et al. (1994) Plant J. 5: 299).

Once the construct of the present invention has been introduced into the genome of a cell, the desired sense or antisense sequence is expressed by the action of the promoter. In the case of inducible or developmentally regulated promoters, expression will not be continuous but rather will depend upon the presence of an inducing agent or achievement of a developmental stage. In cases where a tissue specific promoter has been used, expression will be limited to the particular tissue.

Because, in one embodiment, the constructs of the present invention lack a normal 3′ UTR involved in nuclear export of transcription products, the RNA produced is retained within the nucleus. In another embodiment, the construct includes a self-cleaving ribozyme. The self-cleaving nature of the ribozyme results in the production of free sense or antisense RNA. The inability of the transcripts to be exported from the nucleus, results in relatively high concentrations of the transcripts within the nucleus. As antisense RNA transcripts accumulate in the nucleus, they presumably hybridize to any complementary sense RNA transcripts. Without being bound by theory, it is believed that due to the high concentration of antisense transcripts within the nucleus, it is more likely that sense/antisense hybrids will form than in the cytoplasm where antisense transcripts are present at a lower concentration. It is also believed that the formation of the hybrids results in an inability of the sense RNA (mRNA) to be translated into the encoded protein, but instead the hybrid RNA molecule is eventually degraded within the nucleus. By preventing translation of the mRNA into the encoded protein, the constructs of the present invention result in the effective suppression of the targeted gene or sequence. Furthermore, the presence of sense/antisense hybrids in the nucleus will presumably trigger a sequence-specific RNA degradation pathway, which will further reduce the number of transcripts from the target gene and increase the efficacy of gene down-regulation.

The mechanism by which localization of sense transcripts within the nucleus results in suppression of gene expression is unknown. Without being bound by theory, the mechanism may be similar to that observed with co-suppression. The present invention, however, differs significantly from traditional co-suppression where co-suppression is thought to result from interactions within the cytoplasm, while the present invention relies upon the prevention of nucleus to cytoplasm transport of RNA transcripts.

Once the constructs encompassed by the present invention have been successfully introduced into the genome of a plant, the plant can be replicated using traditional breeding techniques. For example, plants transformed with the sense and/or antisense constructs can be “selfed” to produce homozygous plants. Additionally, plants of the present invention can be crossed to form hybrids containing the sense and/or antisense constructs which can then be selfed to form a homogeneous population. Thus, the present invention encompasses not only plants containing the sense and/or antisense constructs of the present invention, but also seeds, hybrids and uniform populations produced from such plants.

Likewise, once the constructs of the present invention have been introduced into the genome of an animal, such a founder animal can be used to produce offspring by any suitable method and the offspring then inbred to produce a population homozygous for the construct.

### EXAMPLES

The following examples are intended to provide illustrations of the application of the present invention. The following examples are not intended to completely define or otherwise limit the scope of the invention.

### Example 1

### Vectors

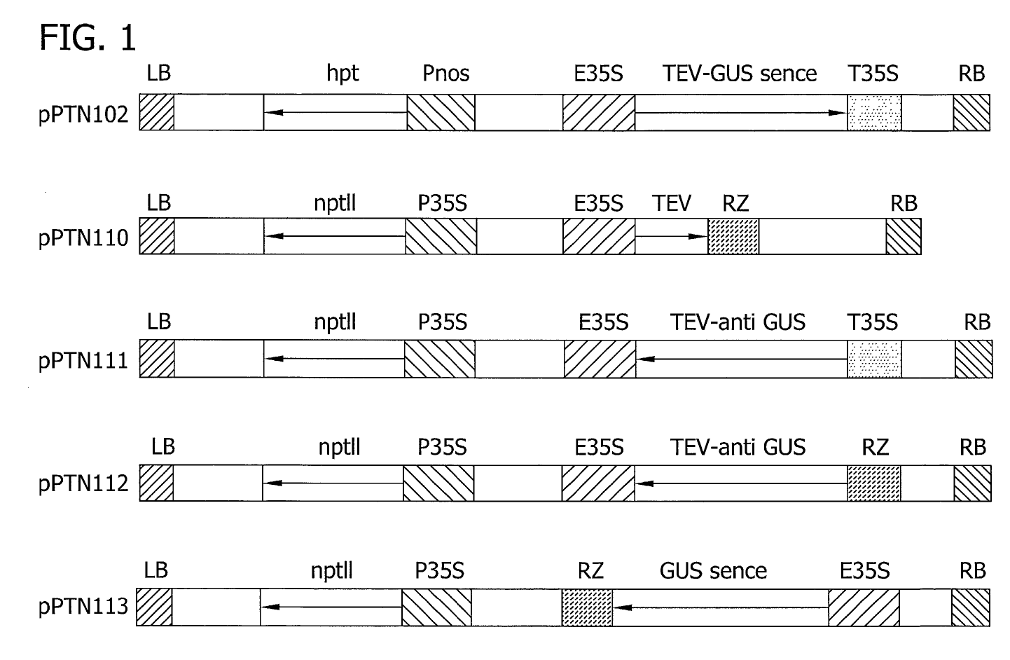

Plasmid cloning was performed using standard procedures (Sambrook et al. Molecular Cloning, 2nd ed., Cold Spring Harbor Laboratory Press, 1989). The binary vector pPTN102 was assembled by ligating the HindIII/EcoRI GUS cassette from pE7131-GUS (Mitsuhara et al. (1996) Plant Cell. Physiol. 37:49) into pGPTV-hpt (Becker et al. (1992) Plant Molec. Biol. 20:1195). The GUS reporter gene was chosen to examine the role of 3′-end modification of transcripts in plants. Four constructs were made with the GUS reporter gene and a fifth construct without GUS served as a negative control (FIG. 1). Plasmid pPTN102 was a binary vector that contained GUS driven by the 35S promoter and ended with a CaMV 35S termination sequence for nuclear export. The GUS gene was preceded by the TEV leader and the W intron, and the GUS cassette was situated between the right border and the hygromycin resistance gene. The polyadenylation signal sequence in the plant expression vector pRTL2 (Carrington and Freed (1990) J. Virol. 64:1590) was replaced by a self-cleaving ribozyme (RZ) (Liu and Carmichael (1994) Molec. Biotechnol. 2:107) from pBS-RZ-2 (a gift from Gordon Carmichael) to yield pPTN106. The GUS open reading frame (ORF) was subcloned into pPTN106 to yield pPTN108 and pPTN109 which harbor ribozyme-terminated antisense GUS and sense GUS, respectively. The vector pPTN107 is a derivative of pRTL2 containing the antisense GUS cassette terminated by a polyadenylation signal. The control and GUS cassettes from pPTN106, pPTN107, pPTN108 and pPTN109 were subcloned as HindIII fragments into pPZP112 (Hajdukiewicz et al. (1994) Plant Mol. Biol. 25:989) to produce the binary vectors pPTN110, pPTN111, pPTN112 and pPTN113. Plasmid pPTN111was the same as pPTN102 except GUS was in the antisense orientation. Plasmid pPTN112 possessed GUS in the antisense orientation, driven by the CaMV 35S promoter and terminated with a cis-acting ribozyme to promote generation of a free 3′ end and to promote nuclear retention, instead of the CaMV 35S termination sequence. For pPTN113, GUS was in the sense orientation and terminated with a ribozyme. Plasmid pPTN110 was a negative control which possessed a TEV leader driven by the 35S promoter and terminated with the cis-cleaving ribozyme. Plasmids pPTN110, pPTN111, pPTN112 and pPTN113 each had the GUS cassette situated between the right border and the kanamycin resistance gene instead of the hygromycin resistance gene used for pPTN102. At least 3 individual transgenic tobaccos were isolated harboring each construct.

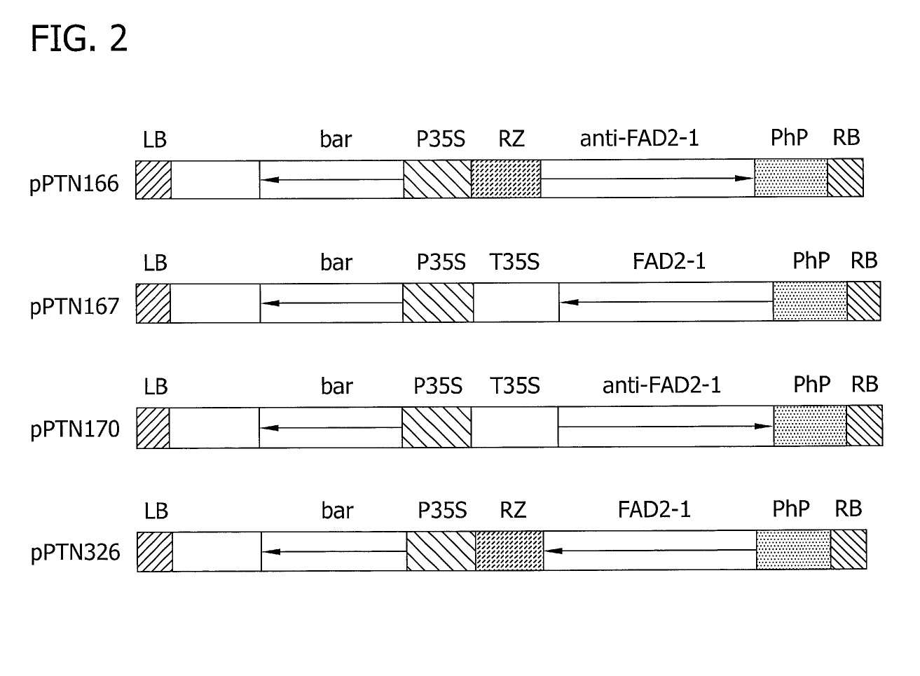

The embryo-specific, FAD2-1 gene from soybean was chosen to determine if the nuclear-localization strategy could be employed for endogenous gene down-regulation. Four binary constructs containing FAD2-1 were made (FIG. 2). The FAD2-1 ORF was isolated via PCR from genomic DNA (soybean genotype A3237). PCR primers were designed to published sequence (Heppard et al. (1996) Plant Physiol. 110:311). The primers were Fad2-5 (5′-ttttttctagaactaggcatgggtctagc-3′; SEQ ID NO: 1) and Fad2-3 (5′-tttttggatccccatcaatacttgttc-3′; SEQ ID NO: 2) with an XbaI site on the 5′ primer and a BamHI site on the 3′ primer. PCR reactions included 200 ng of total genomic soybean DNA as template, 1× PCR buffer (20 mM Tris-HCl, pH=8.4, 50 mM KCl), 0.2 mM of each dNTP, 1.5 mM MgCl₂, 10 ng of each primer and 1 ml of Taq polymerase (Gibco Life Technologies Cat. # 18038-042). PCR conditions were: 1 cycle for 5 minutes at 94° C.; 2 cycles for 1 minute at 94° C., 1 minute at 45° C., 2 minutes at 72° C.; 35 cycles for 1 minute at 94° C., 1 minute at 55° C., 2 minutes at 72° C.; and a 4° C. holding temperature. The PCR product was digested with BamHI and XbaI and subcloned into pBluescript KS+ to yield pPTN156 and then sequenced. Four expression cassettes were assembled with the FAD2-1 ORF. All expression cassettes were under the control of the common bean, embryo-specific b-phaseolin promoter (PhP). FAD2-1 was subcloned downstream of the promoter in either sense or antisense orientation. The antisense cassettes ended with a 3′ termination signal or a self-cleaving ribozyme. The respective cassettes were cloned into a binary vector which carried a bar cassette under the control of the CaMV 35S promoter. The resultant vectors, pPTN166, pPTN167, pPTN170 and pPTN326, harbored the FAD2-1 cassettes in the following orientations: PhP-antisense FAD2-1-RZ, PhP-sense FAD2-1-T35S, PhP-antisense FAD2-1-T35S and PhP-sense FAD2-1-RZ, respectively. Plasmid pPTN166 contained FAD2-1 in antisense orientation driven by the embryo-specific, Phaseolin promoter and terminated with a ribozyme for nuclear localization. Plasmid pPTN167 was a normal co-suppression construct. It contained FAD2-1 in sense orientation driven by the Phaseolin promoter and ended with the CaMV 35S terminator for nuclear export. Plasmid pPTN170 was a normal antisense construct. It was the same as pPTN167 except the FAD2-1 gene was in antisense orientation. Plasmid pPTN326 was the same as pPTN166 except FAD2-1 was in sense orientation. Each of these constructs possessed a bar cassette for glufosinate selection.

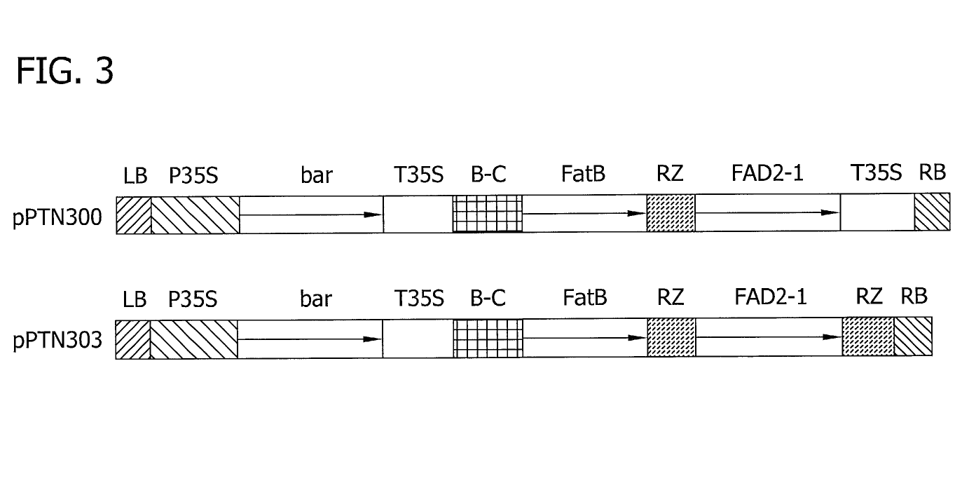

The embryo-specific FatB and FAD2-1 genes were employed to show that the nuclear-localized strategy could be used to down-regulate multiple genes with a single promoter. Two binary constructs, pPTN300 and pPTN303, were made containing FatB and FAD2-1, both in sense orientation and both driven simultaneously by the embryo-specific, b-conglycinin promoter (FIG. 3).

The soybean FatB gene was kindly provided by Tony Kinney of Dupont as clone pBS56 (A. J. Kinney (1997) “Genetic Engineering of oilseeds for Desired Traits” in Genetic Engineering vol. 19, pg. 149-166 (Setlow, J. K. ed.) Plenum Press, N.Y.). An intermediate vector, Fat/RZ (PTN309) harboring the b-conglycinin promoter, FatB sense ORF and ribozyme was made. The b-conglycinin promoter and sense FatB ORF were PCR amplified from pBS56 using a 5′ primer TB-13, 5′-ATTACGAGCTCAAGCTTGATCCATGCCCTTC-3′ (SEQ ID NO: 3), which contains SstI and HindIII sites and is complementary to the upstream promoter sequence, and a 3′ primer TB-14, 5′-AATCGGAATTCAAATCTTAGGTGCTTTC-3′ (SEQ ID NO: 4), which contains an EcoRI site and is complementary to the 3′-end of the FatB ORF including the stop codon. PCR reactions included 100 ng of HindIII-digested pBS56, 1× PCR buffer (20 mM Tris-HCl, pH=8.4, 50 mM KCl), 0.2 mM of each dNTP, 2 mM MgCl₂ and 0.1-1 mg of each primer in 100 ml volume. PCR reactions were mixed, overlaid with 2 drops of mineral oil and brought to 80° C. before adding 0.6 ml of Taq polymerase. PCR reactions included 2 cycles at 94° C. for 1 minute, 45° C. for 30 seconds, 72° C. for 2 minutes followed by 7 cycles at 94° C. for 30 seconds, 55° C. for 30 seconds, 72° C. for 2.5 minutes and then 1 cycle at 72° C. for 5 minutes. The PCR products were digested with SstI/EcoRI and subcloned into TB-RZ (pPTN304) to yield pPTN309. TB-RZ (pPTN304) is a derivative of BS-RZ-2 (Liu and Carmichael (1994) Molec. Biotechnol. 2:107) which was kindly provided by Gordon Carmichael. TB-RZ was generated by HindIII/SalI digestion of BS-RZ-2, followed by mung bean nuclease treatment and self-ligation to remove relevant restriction sites. DNA sequencing confirmed the removal of restriction sites.

Construction of pPTN300 required numerous intermediate vectors. Soybean FAD2-1 was PCR amplified using a 5′ primer TB-15, 5′-AATCGCTCGAGACTAGGCATGGGTCTAGC-3′ (SEQ ID NO: 5), which contains a XhoI site and the ATG start codon of FAD2-1, and a 3′ primer TB-16, 5′-AAATTGGTACCGAGCTCAAGCTTGATTTTGGTTTTAGGAATTAG-3′(SEQ ID NO: 6), which contains KpnI, SstI and HindIII sites and is complementary to downstream sequence of the CaMV35S terminator. PCR reactions included 908 ng of HindIII-digested pPTN167 which harbors the FAD2-1 ORF ended with the CaMV35S terminator. PCR reactions contained 1× buffer, 0.2 mM of each of the 4 dNTPs, 1.5 mM MgCl₂ and 1 mg of each primer. PCR reactions were mixed, overlaid with 2 drops of mineral oil and brought to 80° C. before adding 0.5 ml of polymerase (containing 2.5 U of BRL Taq polymerase and 0.025 U of Pfu polymerase (Stratagene #600153)). PCR reactions included 2 cycles at 94° C. for 30 seconds, 40° C. for 30 seconds, 72° C. for 7 minutes followed by 6 cycles at 94° C. for 30 seconds, 50° C. for 30 seconds, 72° C. for 7 minutes and then a 72° C. extension for 5 minutes. PCR products were digested with XhoI/KpnI and subcloned into pPTN309 to generate FAT sense/RZ/FAD2-1 sense/T35S in KS+(pPTN312). The cassette from pPTN312 was digested with SstI and subcloned into the binary vector pPTN130 to generate pPTN300 (the pertinent sequence arrangement is b-conglycinin promoter- FatB sense- RZ- FAD2-1 sense- CaMV35S terminator).

Construction of pPTN303 also required numerous intermediate vectors. FAD2-1 was PCR amplified using FAD2-3 and FAD2-5 primers described earlier. PCR reactions included 100 ng of FAD2-1 template, 1× buffer, 0.2 mM of each dNTP, 2 mM MgCl₂ and 0.1-1 mg of each primer in 100 ml volume. Reactions were mixed, overlaid with 2 drops mineral oil and brought to 80° C. before adding 0.8 ml of Taq polymerase. PCR reactions included 2 cycles at 94° C. for 1 minute, 45° C. for 30 seconds, 72° C. for 2 minutes followed by 7 cycles at 94° C. for 30 seconds, 55° C. for 30 seconds, 72° C. for 2.5 minutes and then a 72° C. extension for 5 minutes. Amplification products were cut with XbaI/BamHI and subcloned into BS-RZ-2 to yield FAD/BS-RZ-2 (pPTN305). Vector pPTN305 was then used in another round of PCR to generate a sense FAD2-1/RZ sequence with appropriate restriction sites. PCR reactions included a 5′ primer TB-15 (described above) and a 3′ primer TB-17, 5′-AAATTGGTACCGAGCTCGACGGTATCGATAAGCTT-3′ (SEQ ID NO: 7), which contains KpnI, SstI, ClaI and HindIII sites and has identity to the polycloning region of pBluescript. PCR reactions included 624 ng of pPTN305 digested with SstI/XhoI, 1× buffer, 0.2 mM of each dNTP, 1.5 mM MgCl₂, 1 mg of each primer in 100 ml volume. Reactions were mixed, overlaid with 2 drops of mineral oil and brought to 80° C. before adding 0.5 ml of polymerase (containing 2.5 U of BRL Taq polymerase and 0.025 U of Pfu polymerase (Stratagene #600153)). PCR reactions included 2 cycles at 94° C. for 30 seconds, 40° C. for 30 seconds, 72° C. for 7 minutes followed by 6 cycles at 94° C. for 30 seconds, 50° C. for 30 seconds, 72° C. for 7 minutes and then a 72° C. extension for 5 minutes. PCR products were digested with XhoI/KpnI and sub-cloned into pPTN309 to generate FAT sense/RZ/FAD2-1 sense/RZ in KS+ (pPTN313). The cassette from pPTN313 was digested with SstI and subcloned into the binary vector pPTN130 to generate pPTN303 (the pertinent sequence arrangement is b-conglycinin promoter- FatB sense- RZ- FAD2-1 sense- RZ).

Plasmids pPTN300 and pPTN303 both contain the cis-cleaving ribozyme as a linker between the 2 genes as well as a bar cassette for glufosinate selection. The only difference between the 2 binaries was that plasmid pPTN300 was ended with a CaMV 35S terminator and plasmid pPTN303 was terminated with a ribozyme.

### Example 2

### Tobacco and Soybean Transformations

Binary vectors were mobilized into Agrobacterium tumefaciens strain C58C1 (Koncz and Schell (1986) Mol. Gen. Genet. 204:383) by triparental matings (Ditta et al. (1980) Proc. Natl. Acad. Sci. USA 77:7347). The Agrobacterium transconjugant carrying pPTN102 was selected on LB plates supplemented with 50 mg/L rifampicin, 50 mg/L gentamicin and 50 mg/L kanamycin. Agrobacterium transconjugants carrying pPTN110, pPTN111, pPTN112 and pPTN113 were selected on LB supplemented with 50 mg/L rifampicin, 50 mg/L gentamicin and 75 mg/L chloramphenicol. Agrobacterium transconjugants carrying pPTN166, pPTN167, pPTN170, pPTN326, pPTN300 and pPTN303 were selected on LB supplemented with 50 mg/L rifampicin, 50 mg/L gentamicin, 100 mg/L streptomycin and 100 mg/L spectinomycin. Binary vectors in Agrobacterium were confirmed by plasmid preps and restriction digestion (Sambrook et al., Molecular Cloning, 2nd ed., Cold Spring Harbor Laboratory Press, 1989).

Tobacco (cultivar Xanthi) transformations were conducted via the leaf disc method described by Horsch et al. (1985) Science 227:1229. Transformants were selected on 10 mg/L hygromycin (pPTN102) or 150 mg/L kanamycin (pPTN110, pPTN111, pPTN112 or pPTN113). Soybean transformations were conducted as described by Zhang et al. (1999) Plant Cell, Tissue and Organ Culture 56:37) using the cotyledonary-node transformation system (Hinchee et al. (1988) Bio/Technol. 6:915; Clemente et al. (2000) Crop Sci. 40:797). Transformants were selected on 5 mg/L glufosinate during shoot initiation and 3 mg/L during shoot elongation steps.

### Example 3

### RNA Isolation and Northern Blottinq

Comparison of the CaMV 35S terminator and the cis-acting ribozyme with respect to sub-cellular transcript localization was made using transgenic tobacco harboring GUS constructs (FIG. 1). Evaluation of the foreign GUS gene alleviated potential problems with gene silencing or endogenous gene expression. To ascertain the sub-cellular location of transcripts produced by the different clones, total RNA and nuclei RNA were isolated from approximately 2 week old seedlings. A comparison of total RNA to nuclei RNA was made because RNA isolation from these compartments was much simpler and efficient compared to isolation of cytoplasmic RNA from plants. Furthermore, the data derived from these experiments was much simpler to standardize and interpret compared to isolation of cytoplasmic RNA. This is because it is difficult to assess the amount of RNA that is present from broken nuclei when using cytoplasmic RNA samples and isolation of cytoplasmic RNA requires inconsistent and lengthy protocols.

Transgenic GUS tobacco seeds were sterilized by suspending approximately 200-500 seeds in 1 ml of 50% Clorox®, 0.02% Triton X-100 with constant agitation for 5 minutes at room temperature (RT). Samples were then spun at 735× g for 5 seconds and the Clorox® solution removed with a pipette. Seeds were then resuspended in 1 ml of sterile deionized distilled water (ddH₂O) with constant agitation for 1 minute at room temperature followed by a brief 735× g spin and removal of the water. Seeds were washed with ddH₂O a total of 4 times. Each sample was then suspended in 1 ml of ddH₂O and transferred to a tube with 9 ml of 0.2% Type A agar amended with 100 mg cefotaxime/L. This was overlaid onto 150 mm×15 mm petri plates containing selection medium: 0.5× Murashige-Skoog salts and 1× Fe-ethylene diamine tetraacetic acid (EDTA) (Murashige and Skoog (1962) Physiol. Plant 15:473), 1% sucrose, pH=5.7, 0.7% Type A agar, 1× Gamborg B5 vitamins (Gamborg et al. (1968) Exp. Cell Res. 50:151) amended with 15 mg hygromycin/L and/or 100 mg kanamycin/L. Plates were wrapped with Parafilm® and incubated in a 24° C. growth chamber with a 16 hour light/8 hour dark photoperiod.

After a 2-3 week selection period, numerous whole seedlings were harvested and weighed. Seedlings were then subjected to RNA isolation. For intact RNA isolation, 100 mg of seedlings was processed with 0.75 ml Trizol LS (Life Technologies, Inc., GibcoBRL, Rockville, MD) plus 0.1 ml ddH₂O according to the manufacturer's instructions. Two exceptions to the manufacturer's protocol included an additional chloroform extraction prior to isopropanol precipitation and RNA suspension in 100% formamide.

For total RNA and nuclei RNA isolation, seedlings were ground in an ice-cold mortar with a glycerol extraction buffer (10 mM HEPES, pH=8.0, 40% glycerol, 5 mM MgCl₂, 0.1 mM EDTA) at a ratio of 300 mg tissue per 1 ml of ice-cold extraction buffer. For total RNA isolation, 0.25 ml of the homogenate was mixed with 0.75 ml of Trizol LS. For nuclear RNA isolation, the remaining homogenate was filtered through 1 layer of 33 mM nylon mesh (pre-washed with ddH₂O and autoclaved) into a centrifuge tube on ice. The filtrate was spun in Eppendorf tubes at 1,310× g for 30-60 seconds. The supernatant was removed with a pipette. The crude nuclei/organelle pellet was then resuspended in 1 ml of cold extraction buffer by gentle inversion. Samples were next spun at 1,310× g for 30-60 seconds and the supernatant was removed with a pipette. At this point, the crude nuclei pellet was free from cell walls, cytoplasmic components, and broken organelles. The resulting pellet(s) was suspended in 0.25 ml of cold extraction buffer giving a total volume of 0.3-0.35 ml. The crude nuclei suspension was then mixed with 1.1 ml of Trizol LS. RNA extractions were as above.

For Northern analysis 10 mg of RNA or 3 ml of RNA marker (Life Technologies, Inc., GibcoBRL, Rockville, Md., cat # 15260-016) was combined with formamide, 1 ml of ethidium bromide (10 mg per ml), 0.45 ml formaldehyde, and 2 ml of 10× MESA buffer (0.4M MOPS, pH=7.0, 0.1M NaAc, 10 mM EDTA) in 18 ml volume. Samples were incubated at 75° C. for 5 minutes and then set on ice. Next, 2 ml of 10× stop dye (0.05% bromophenol blue, 40% sucrose, 0.1M EDTA, 0.5% sodium dodecyl sulfate (SDS)) was added. Samples were loaded onto a 1% agarose, 1× buffer (1× MESA buffer, 0.3M formaldehyde) gel and ran in 1× buffer at 1.5-2.5 V/cm for up to 6 hours. Gels were washed by immersing 2 times in ddH₂O and then 2 times in 2× SSC (Sambrook et al. Molecular Cloning, 2nd ed., Cold Spring Harbor Laboratory Press, 1989). Washes were at room temperature with gentle agitation for 10 minutes each. RNA was transferred to positive nylon membranes (BioRad Laboratories, Hercules, Calif.) using 20× SSC for 12-18 hours and subsequently fixed via UV irradiation. Filters were hybridized in 25 ml of 7% SDS, 0.5M Na₂HPO₄, pH=7.2, 1 mM EDTA, 1% bovine serum albumin (BSA) amended with 10-15 ng of denatured probe. Probes were labeled with [a-³²P]dCTP using the random priming kit from Stratagene (La Jolla, Calif.). Hybridizations were incubated 14-20 hours at 65° C. Filters were washed under high stringency including 2 washes of 0.2× SSC, 0.1% SDS at 65° C. for 10 minutes each. Filters were exposed to film with intensifying screens at −80° C.

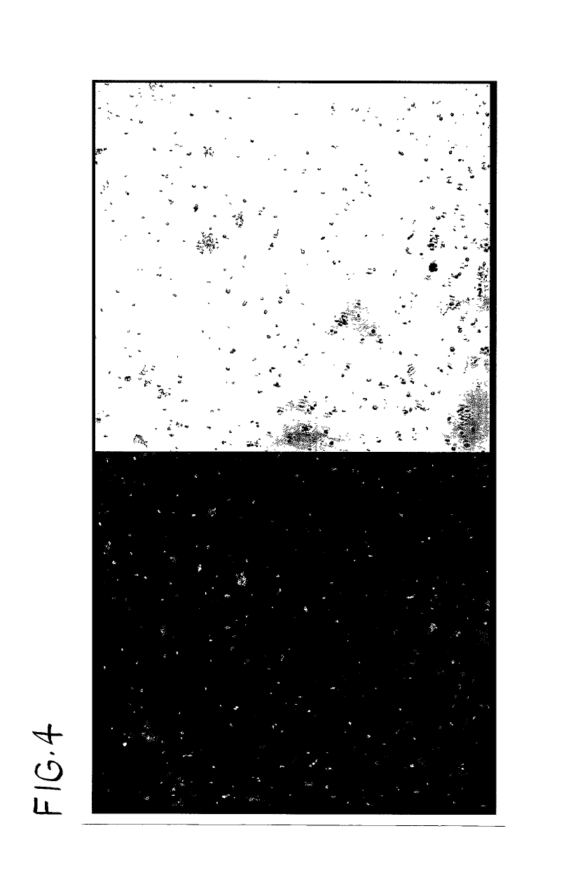

The results are shown in FIG. 4. Nuclei staining in FIG. 4 shows that good nuclei were isolated. The large red bodies are intact nuclei. The smaller, yellow-green bodies are chloroplasts. These crude nuclei (organelle) fractions were filtered and washed free of cytoplasmic components. This contrasts with total nuclei RNA preps which contain nuclei RNA, organelle RNA, and all cytoplasmic RNA regardless of the RNA location within or among the plant cells. Thus nuclei RNA samples have a much higher concentration of nuclei RNA than total RNA samples.

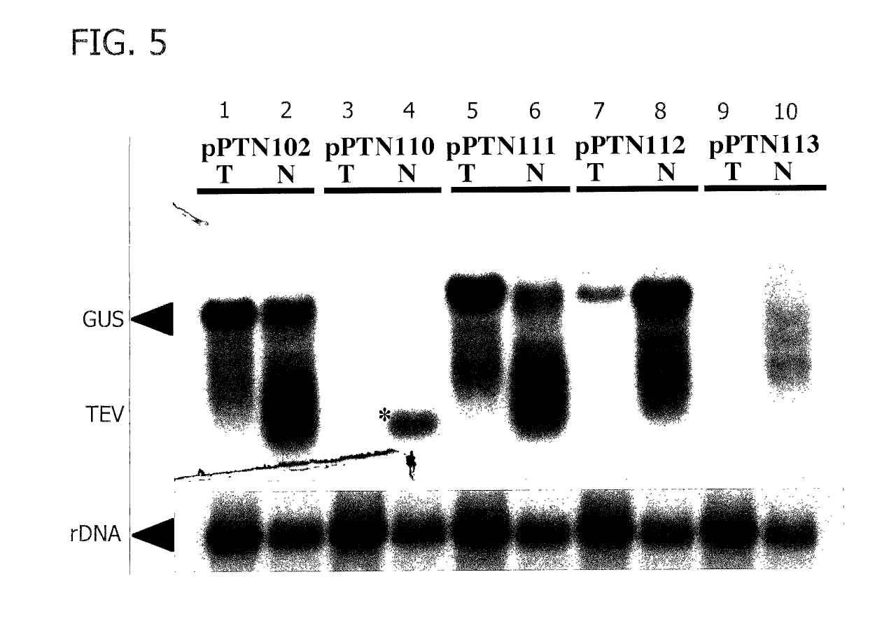

A TEV leader-GUS probe was used for Northern analysis in FIG. 5. GUS expression for pPTN113 was very low and this was observed for numerous individuals harboring pPTN113. The Northerns showed much higher levels of total RNA than nuclear RNA for clones pPTN102 and pPTN111. Subtraction of nuclear RNA from total RNA implied that the majority of transcripts generated by these 2 clones resided outside of the nucleus. The opposite hybridization pattern was seen for clones pPTN110, pPTN112 and pPTN113. These ribozyme-terminated constructs yielded transcripts which appeared to accumulate exclusively in the nucleus. The smaller molecular weight for the signal seen for pPTN110 was exclusive to pPTN110 since it is the only construct that expressed a TEV leader but no FAD2-1 ORF.

### Example 4

### In Situ Hybridization

All procedures were carried out at RT unless indicated otherwise. Root tips, taken from 2 week-old seedlings grown on agar selection medium as described above, were fixed in 4% paraformaldehyde in PBS (Sambrook et al., Molecular Cloning, 2nd ed., Cold Spring Harbor Laboratory Press, 1989) for 2 hours. Roots were washed twice in PBS for 10 minutes each, once in PBS amended with 1 mg/ml sodium borohydride for 10 minutes, and once in PBS plus 0.2% Triton X-100 for 15 minutes. Samples were incubated in PBS containing 30% sucrose overnight at 4° C. Frozen sections (8 mM in thickness) were collected on poly-L-lysine coated slides, air dried and stored at −20° C. overnight before use. Slides containing sample sections were treated with 0.2 N HCl for 5 minutes, proteinase-K (5 ug/ml in TE (10 mM Tris, 1 mM EDTA, pH=8.0)) for 10-15 minutes at 37° C., and 0.25% acetic anhydride plus 0.1M triethanolamine for 10 minutes. Samples were incubated in prehybridization buffer (5× SSC, 1× Denhardt's solution, 1% SDS, 50 mg/ml denatured salmon sperm DNA, 50% formamide, 20 mM sodium phosphate, pH=6.6) for 2 hours at 50° C. followed by an overnight hybridization at 50° C. in the same buffer plus 10% dextran sulfate and GUS probe (1-2 mg/ml). GUS probes were antisense or sense-specific, DIG-labeled RNA probes which were polymerized and then hydrolyzed to less than 500 base pairs using the DIG labeling system from Roche Molecular Biochemicals Inc. (Indianapolis, Ind.). After washing with 2× SSC at 60° C. for 10 minutes and 37° C. for 10 minutes, slides were treated with 0.2 mg/ml RNAase A in 2× SSC, followed by a series of washes with SSC. The final wash was with 0.2× SSC at 37° C. for 30 minutes. Samples were rinsed once in TTBS (0.05% Tween 20, 1× TBS (20 mM Tris, 0.5M NaCl, pH=7.5)) and then incubated with TTBS plus 5% BSA (Fraction-V) for 1 hour. Slides were then incubated in TTBS containing 1% BSA and FITC-conjugated sheep anti-DIG antibodies (1:5 dilution, Roche Molecular Biochemicals, Inc.) for 2 hours. After 2 washes with TTBS and 1 wash with TBS (10 minutes each), the slides were stained with 10 mg/ml propidium iodide (DNA-staining) for 5 minutes, rinsed in TBS, and mounted immediately for image collection. Images were collected with a BioRad MRC1024ES confocal laser scanning microscope using a dual excitation (488/568 nm) and dual emission (520/598 nm) program.

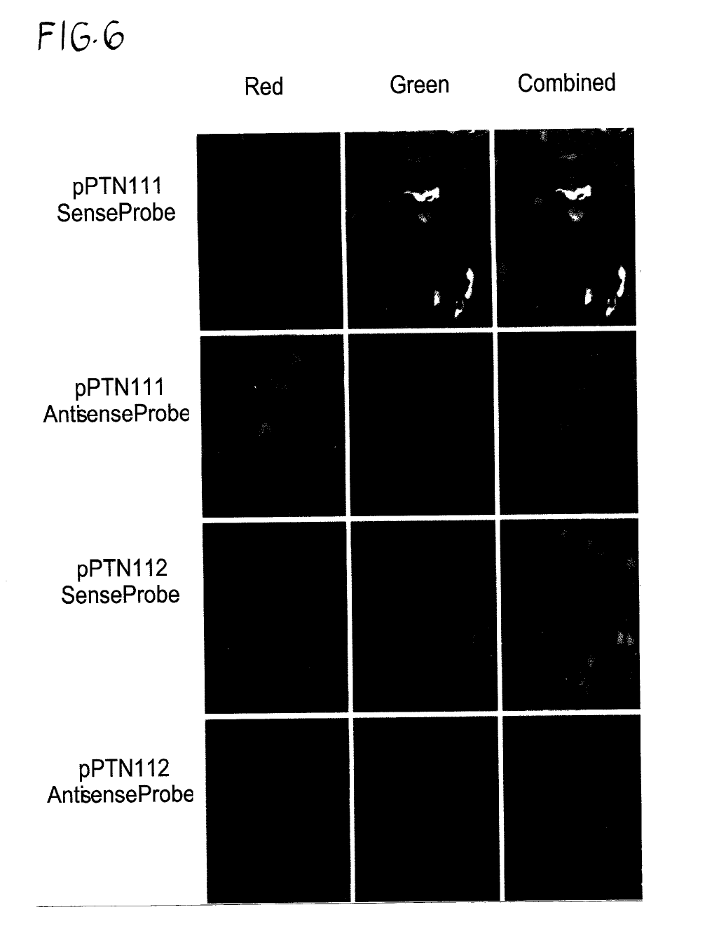

In situ hybridization using strand-specific GUS probes and subcellular microscopy corroborated the Northern results. FIG. 6 shows hybridization results for transgenic individuals harboring pPTN111 or pPTN112 which expressed cytoplasm-localized GUS and nuclear-localized GUS, respectively. The red channel showed nuclei staining and the green channel showed in situ hybridization of strand-specific probes. Comparing the green channel of pPTN111 sense and antisense showed a much more intense, disperse signal with the sense probe although some background was present with the antisense probe. The combined channel shows yellowing of nuclei using the sense probe but not the antisense probe indicating specific hybridization by the sense probe. This was expected since some of the GUS RNA should still be in the nuclei for pPTN111 transgenics even though most of the RNA had been exported out of the nucleus. Comparing the green channel of pPTN112 sense and antisense showed staining of nuclei that was specific only for the sense probe. There was some background emanating from what appeared to be cell structure. Again, the combined channel showed yellowing of nuclei for the sense probe indicating specific binding by the sense probe but not the antisense probe. Comparison of pPTN111 and pPTN112 showed that the sense probe was disperse in the pPTN111sample and concentrated in the nucleus for pPTN112.

### Example 5

### Fatty Acid Analysis

Seeds or seed chips for 4 to 8 T1, T2 or T3 generation of transgenic soybeans were used for fatty acid analysis. Fatty acid analysis was performed using gas chromatography according to the procedure of Butte et al. ((1982) Analytical Lett., 15(A10):841-850).

Transgenic soybeans were selected with glufosinate, and the presence of a T-DNA was confirmed by Southern analysis (data not shown). Fatty acid analysis was performed on up to 8 seed chips from each T1 transgenic (Table 1). A high oleic acid phenotype was observed for 3 of 4 transgenic soybeans harboring nuclear-localized antisense FAD2-1 (pPTN166). Transgenics 1-3, 1-5 and 1-6 yielded an average of 63.0±6.2% oleic acid compared to 13.7% for wild-type. A high oleic acid phenotype was observed for 2 of 9 transgenic soybeans harboring cytoplasm-localized antisense FAD2-1 (pPTN170). Transgenics 3-1 and 3-2 yielded an average of 83.0±1.1% oleic acid compared to 13.7% for wild-type. Two of the eight soybean transformants carrying the sense nuclear-localized FAD2-1 (pPTN326) displayed an elevated oleic acid phenotype as shown in Table 1. Soybean transformants 6-1, and 6-2 possessed oleic acid levels of 77.2% and 77.3% respectively. As expected, the high oleate acid phenotype came primarily at the expense of polyunsaturates (18:2+18:3), although there was also a noticeable decrease in saturates (16:0+18:0). There were no high oleate transgenics harboring the normal cosuppression construct pPTN167.

Transgenic soybeans with dual gene constructs were selected with glufosinate and the presence of a T-DNA was confirmed by Southern analysis (data not shown). Fatty acid analysis was performed on up to 8 seed chips from each T1 transgenic (Table 2). A high oleate acid phenotype was observed for 6 of 15 transgenic soybeans harboring pPTN300. Five of these transgenics 4-3, 4-6, 4-14 and 4-15 yielded an average of 66.2±11.0% oleic acid compared to 13.7% for wild-type. The decrease in polyunsaturates (18:2±18:3) indicated down-regulation of the FAD2-1 desaturase. Transgenic 4-2 was the only pPTN300 transgenic with a highly significant decrease in saturated fatty acids (16:0+18:0) in addition to a large increase in oleic acid. This transgenic had 5.7% saturates compared to 15.8% for wild-type which indicated that the FatB thioesterase gene had been down-regulated, while the increase in oleic acid and decrease in polyunsaturates was the result of down-regulated FAD2-1. A high oleate phenotype was observed for 5 of 27 transgenic soybeans harboring pPTN303. Four of the five pPTN303 transgenics (5-3, 5-5, 5-10 and 5-24) yielded an average of 89.2%±1.6% compared to 13.7% for wild-type. The great decrease in polyunsaturates and increase in oleic acid indicated strong FAD2-1 down-regulation. Importantly, the saturated fatty acid composition (16:0+18:0) of these four pPTN303 transgenics was decreased to an average of 5.2%±0.3% compared to 15.8% for wild-type indicating FatB thioesterase down-regulation in all 3 transgenics. Thus, the ribozyme-terminated pPTN303 construct produced a very dramatic phenotype and was more effective for down regulating multiple genes at one time compared to pPTN300.

### Conclusion

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventors do not intend to be bound by those conclusions and functions, but put them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

TABLE 1
Fatty Acid Profiles of Transgenic Soybeans
Harboring Vectors PTN166, PTN167 or PTN170

| No. | Event | Vector | 16:0 | 18:0 | 18:1 | 18:2 | 18:3 |
|---|---|---|---|---|---|---|---|
| WT | Wild-type | — | 11.8 | 4.0 | 13.7 | 59.3 | 11.2 |
| 1-3 | 291-5 T2 (R1-6) | PTN166 | 10.0 | 3.0 | 62.3 | 16.1 | 8.6 |
| 1-4 | 280-13-2 T3 | PTN166 | 11.1 | 3.5 | 17.8 | 58.8 | 8.8 |
| | (R2-11) | | | | | | |
| 1-5 | 280-13 | PTN166 | 10.3 | 2.7 | 57.2 | 19.4 | 10.4 |
| 1-6 | 288-9B | PTN166 | 8.4 | 3.9 | 69.5 | 10.3 | 7.9 |
| 2-1 | 265-1A T2 (R1-3) | PTN167 | 11.6 | 3.4 | 15.7 | 59.1 | 10.2 |
| 2-2 | 269-9 T2 (R1-3) | PTN167 | 12.7 | 4.7 | 16.8 | 59.1 | 6.7 |
| 2-3 | 309-14 T2 (R1-3) | PTN167 | 11.4 | 3.5 | 17.8 | 58.5 | 8.8 |
| 2-4 | 283-12 T3 (R2-1) | PTN167 | 11.2 | 3.5 | 17.4 | 58.9 | 9.0 |
| 2-5 | 309-63 | PTN167 | 11.8 | 3.5 | 17.2 | 57.8 | 9.7 |
| 2-7 | 283-11 (R1-3) | PTN167 | 11.9 | 4.1 | 15.9 | 57.1 | 11.0 |
| 2-8 | 318-40 | PTN167 | 11.8 | 3.1 | 18.6 | 57.7 | 8.8 |
| 2-9 | 322-19 | PTN167 | 11.5 | 3.7 | 19.7 | 57.2 | 7.9 |
| 3-1 | 294-14 | PTN170 | 7.2 | 4.2 | 82.2 | 3.3 | 3.1 |
| 3-2 | 294-5 T2 (R1-15) | PTN170 | 7.5 | 3.5 | 83.8 | 2.2 | 3.0 |
| 3-3 | 306-1 T2 (R1-3) | PTN170 | 11.8 | 3.7 | 17.9 | 56.5 | 10.1 |
| 3-4 | 309- T2 (R1-1) | PTN170 | 11.4 | 3.3 | 18.8 | 57.8 | 8.7 |
| 3-5 | 312-08 T2 (R1-2) | PTN170 | 12.2 | 3.3 | 18.0 | 56.8 | 9.7 |
| 3-6 | 306-17 T2 (R1-8) | PTN170 | 11.9 | 3.2 | 18.9 | 56.6 | 9.4 |
| 3-8 | 306-48 | PTN170 | 11.9 | 3.5 | 17.7 | 57.3 | 9.6 |
| 3-9 | 312-38 | PTN170 | 12.3 | 4.0 | 16.5 | 56.9 | 10.3 |
| 3-10 | 312-39 | PTN170 | 12.2 | 4.0 | 18.6 | 56.2 | 9.0 |
| 6-1 | 374-1 | pPTN326 | 7.7 | 3.6 | 77.2 | 4.8 | 6.4 |
| 6-2 | 374-4 | pPTN326 | 7.9 | 3.5 | 77.3 | 4.4 | 6.7 |
| 6-3 | 374-15 | pPTN326 | 12.0 | 3.8 | 15.9 | 55.7 | 11.0 |
| 6-4 | 374-17 | pPTN326 | 11.9 | 4.0 | 17.4 | 56.1 | 10.5 |
| 6-5 | 374-13 | pPTN326 | 11.7 | 3.8 | 16.7 | 56.2 | 11.7 |
| 6-6 | 368-2 | pPTN326 | 11.5 | 3.8 | 20.1 | 53.7 | 11.0 |
| 6-7 | 368-3 | pPTN326 | 11.0 | 3.7 | 16.4 | 57.0 | 12.0 |
| 6-8 | 368-3 | pPTN326 | 11.6 | 3.8 | 17.3 | 56.3 | 11.0 |

TABLE 2
Fatty Acid Profiles of Transgenic Soybeans
Harboring Vectors PTN300 or PTN303

| No. | Event | Vector | 16:0 | 18:0 | 18:1 | 18:2 | 18:3 |
|---|---|---|---|---|---|---|---|
| WT | Wild-type | — | 11.8 | 4.0 | 13.7 | 59.3 | 11.2 |
| 4-1 | 327-18 T1 | PTN300 | 11.7 | 3.4 | 21.1 | 55.1 | 8.7 |
| 4-2 | 335-12 | PTN300 | 2.7 | 3.0 | 89.1 | 2.3 | 2.9 |
| 4-3 | 333-3 | PTN300 | 11.7 | 3.5 | 69.6 | 8.2 | 7.0 |
| 4-4 | 325-65 | PTN300 | 12.6 | 3.4 | 12.9 | 59.3 | 11.8 |
| 4-5 | 327-37 | PTN300 | 15.2 | 4.4 | 15.0 | 55.3 | 10.1 |
| 4-6 | 333-51 | PTN300 | 10.2 | 4.2 | 73.8 | 5.5 | 6.3 |
| 4-7 | 335-4 | PTN300 | 12.6 | 4.1 | 14.1 | 58.6 | 10.6 |
| 4-8 | 327-51B | PTN300 | 14.1 | 4.4 | 12.8 | 57.4 | 11.3 |
| 4-9 | 327-51 | PTN300 | 14.3 | 3.5 | 10.2 | 59.5 | 12.5 |
| 4-10 | 325-1 | PTN300 | 12.1 | 3.2 | 14.2 | 58.9 | 11.6 |
| 4-11 | 333-8 | PTN300 | 11.5 | 3.7 | 72.1 | 5.8 | 6.9 |
| 4-12 | 342-4 | PTN300 | 13.1 | 4.9 | 14.3 | 58.5 | 9.2 |
| 4-13 | 342-5 | PTN300 | 12.7 | 4.3 | 15.6 | 58.8 | 8.6 |
| 4-14 | 333-26 | PTN300 | 9.8 | 4.2 | 46.9 | 30.2 | 8.9 |
| 4-15 | 333-2 | PTN300 | 11.7 | 3.6 | 68.4 | 8.6 | 7.7 |
| 5-1 | 329-38 | PTN303 | 15.7 | 4.0 | 15.4 | 53.7 | 11.2 |
| 5-2 | 329-10 | PTN303 | 12.1 | 3.8 | 17.2 | 58.1 | 8.8 |
| 5-3 | 333-7 | PTN303 | 2.5 | 2.8 | 89.4 | 2.7 | 2.6 |
| 5-4 | 329-61 | PTN303 | 14.4 | 3.9 | 13.8 | 56.6 | 11.3 |
| 5-5 | 329-39 | PTN303 | 3.0 | 2.6 | 87.2 | 2.7 | 4.5 |
| 5-6 | 337-1 | PTN303 | 11.9 | 3.7 | 14.2 | 59.1 | 11.1 |
| 5-7 | 329-11 | PTN303 | 12.5 | 3.6 | 13.5 | 59.8 | 10.6 |
| 5-8 | 325-62 | PTN303 | 12.5 | 3.5 | 15.7 | 57.8 | 10.5 |
| 5-9 | 333-23 | PTN303 | 12.2 | 4.1 | 13.3 | 59.9 | 10.5 |
| 5-10 | 335-13 | PTN303 | 2.2 | 2.6 | 91.1 | 2.0 | 2.1 |
| 5-11 | 335-22 | PTN303 | 12.5 | 4.3 | 18.7 | 54.3 | 10.2 |
| 5-12 | 329-18 | PTN303 | 13.5 | 3.1 | 15.9 | 56.6 | 10.9 |
| 5-13 | 335-38 | PTN303 | 8.1 | 3.8 | 68.8 | 12.4 | 6.9 |
| 5-14 | 329-16 | PTN303 | 12.5 | 3.8 | 13.9 | 58.5 | 11.3 |
| 5-15 | 331-2 | PTN303 | 12.2 | 4.6 | 14.4 | 58.5 | 10.3 |
| 5-16 | 333-28 | PTN303 | 12.7 | 4.5 | 14.7 | 58.1 | 10.0 |
| 5-17 | 337-6 | PTN303 | 12.7 | 4.1 | 13.2 | 60.2 | 9.8 |
| 5-18 | 333-27 | PTN303 | 12.1 | 4.0 | 13.3 | 60.5 | 10.1 |
| 5-19 | 333-10 | PTN303 | 12.5 | 4.0 | 15.4 | 57.0 | 11.1 |
| 5-20 | 329-9 | PTN303 | 13.0 | 4.0 | 14.0 | 57.4 | 11.6 |
| 5-21 | 329-40 | PTN303 | 12.2 | 3.8 | 14.2 | 58.7 | 11.1 |
| 5-22 | 341-8 | PTN303 | 15.7 | 3.9 | 12.7 | 59.1 | 8.6 |
| 5-23 | 341-4 | PTN303 | 17.8 | 3.8 | 11.7 | 58.2 | 8.5 |
| 5-24 | 325-61 T1-2 | PTN303 | 2.9 | 2.3 | 89.1 | 2.5 | 3.2 |
| 5-25 | 335-30 | PTN303 | 13.5 | 3.8 | 11.2 | 60.2 | 11.3 |
| 5-26 | 341-6 | PTN303 | 13.4 | 3.5 | 13.8 | 58.7 | 10.6 |
| 5-27 | 341-3 | PTN303 | 12.9 | 3.8 | 13.3 | 59.4 | 10.6 |

What is claimed is:

1. A method for suppressing gene expression in a eukaryotic cell comprising, transforming said cell with a recombinant construct comprising, a promoter functional in said cell operatively linked to a sense nucleotide sequence of a gene to be suppressed, wherein nucleus-to-cytoplasm transport of transcription products of said construct is inhibited.

2. The method of claim 1, wherein said inhibition of nucleus-to-cytoplasm transport is due to a lack of a normal 3′ UTR.

3. The method of claim 1, wherein said inhibition of nucleus-to-cytoplasm transport is due to a lack of a 3′ terminal sequence.

4. The method of claim 1, wherein said construct further comprises at least one self cleaving ribozyme.

5. The method of claim 1, wherein said promoter is selected from the group consisting of a constitutive promoter, an inducible promoter, a tissue specific promoter, and a developmentally regulated promoter.

6. The method of claim 1, wherein said construct further comprises at least one additional sense nucleotide sequence of at least one additional gene operatively linked to said promoter.

7. A method for suppressing gene expression in a eukaryotic cell comprising, transforming said cell with a recombinant construct comprising, a promoter functional in said cell operatively linked to a plurality of antisense nucleotide sequences of a gene or genes to be suppressed, wherein nucleus-to-cytoplasm transport of transcription products of said construct is inhibited.

8. The method of claim 7, wherein said inhibition of nucleus-to-cytoplasm transport is due to a lack of a normal 3′ UTR.

9. The method of claim 7, wherein said inhibition of nucleus-to-cytoplasm transport is due to a lack of a 3′ terminal sequence.

10. The method of claim 7, wherein said construct further comprises at least one self cleaving ribozyme.

11. The method of claim 7, wherein said promoter is selected from the group consisting of a constitutive promoter, an inducible promoter, a tissue specific promoter, and a developmentally regulated promoter.

12. A method for suppressing expression of a gene in a plant cell, comprising transforming said plant cell with a recombinant construct comprising, a promoter functional in said plant cell, and an antisense nucleotide sequence for the gene to be suppressed, wherein nucleus-to-cytoplasm transport of transcription products of said construct is inhibited.

13. The method of claim 12, wherein said inhibition of nucleus-to-cytoplasm transport is due to a lack of a normal 3′ UTR.

14. The method of claim 12, wherein said construct further comprises at least one self cleaving ribozyme.

15. The method of claim 12, wherein said promoter is selected from the group consisting of a constitutive promoter, an inducible promoter, a tissue specific promoter, and a developmentally regulated promoter.

16. A recombinant vector comprising, a promoter functional in a eukaryotic cell operatively linked to a nucleotide sequence selected from the group consisting of:
a) at least one sense nucleotide sequence of at least one gene to be suppressed,
b) a plurality of antisense nucleotide sequences of at least one gene to be suppressed, and
c) at least one antisense nucleotide sequence for at least one gene to be suppressed;
wherein nucleus-to-cytoplasm transport of transcription products of said at least one nucleotide sequence is inhibited.

17. The recombinant vector of claim 16, wherein said inhibition of nucleus-to-cytoplasm transport is due to a lack of a normal 3′ UTR.

18. The recombinant vector of claim 16, wherein said construct further comprises at least one self cleaving ribozyme.

19. The recombinant vector of claim 16, wherein said promoter is selected from the group consisting of a constitutive promoter, an inducible promoter, a tissue specific promoter, and a developmentally regulated promoter.

20. A host cell comprising the recombinant vector of claim 16.

21. A eukaryotic cell whose genome includes a recombinant construct comprising, a promoter functional in said eukaryotic cell operatively linked to at least one sense nucleotide sequence of at least one gene to be suppressed, wherein nucleus-to-cytoplasm transport of transcription products of said construct is inhibited.

22. The eukaryotic cell of claim 21, wherein said inhibition of nucleus-to-cytoplasm transport is due to a lack of a 3′ terminal sequence.

23. The eukaryotic cell of claim 21, wherein said inhibition of nucleus-to-cytoplasm transport is due to a lack of a normal 3′ UTR.

24. The eukaryotic cell of claim 21, wherein said construct further comprises at least one self cleaving ribozyme.

25. The eukaryotic cell of claim 21, wherein said promoter is selected from the group consisting of a constitutive promoter, an inducible promoter, a tissue specific promoter, and a developmentally regulated promoter.

26. A plant comprising at least one cell of claim 21.

27. A eukaryotic cell whose genome includes a recombinant construct comprising, a promoter functional in said eukaryotic cell operatively linked to a plurality of antisense nucleotide sequences of at least one gene to be suppressed, wherein nucleus-to-cytoplasm transport of transcription products of said construct is inhibited.

28. The eukaryotic cell of claim 27, wherein said inhibition of nucleus-to-cytoplasm transport is due to a lack of a normal 3′ UTR.

29. The eukaryotic cell of claim 27, wherein said inhibition of nucleus-to-cytoplasm transport is due to a lack of a 3′ terminal sequence.

30. The eukaryotic cell of claim 27, wherein said construct further comprises at least one self cleaving ribozyme.

31. The eukaryotic cell of claim 27, wherein said promoter is selected from the group consisting of a constitutive promoter, an inducible promoter, a tissue specific promoter, and a developmentally regulated promoter.

32. A plant comprising at least one cell of claim 27.

33. A plant cell whose genome includes a recombinant construct comprising, a promoter functional in said plant cell operatively linked to at least one antisense sequence for at least one gene to be suppressed; wherein nucleus-to-cytoplasm transport of transcription products of said construct is inhibited.

34. The plant cell of claim 33, wherein said inhibition of nucleus-to-cytoplasm transport is due to a lack of a normal 3′ UTR.

35. The plant cell of claim 33, wherein said inhibition of nucleus-to-cytoplasm transport is due to a lack of a 3′ terminal sequence.

36. The plant cell of claim 33, wherein said construct further comprises at least one self cleaving ribozyme.

37. The plant cell of claim 33, wherein said promoter is selected from the group consisting of a constitutive promoter, an inducible promoter, a tissue specific promoter, and a developmentally regulated promoter.

38. A plant comprising at least one cell of claim 33.

39. A seed from the plant of claim 38.

40. The progeny of the plant of claim 38.
